(19) United States
(12) Patent Application Publication
(10) Pub. No.: US 2022/0073572 A1
(43) Pub. Date: Mar. 10, 2022

(54) PROCESS FOR EXTRACTING PHYCOCYANINS

(71) Applicant: FERMENTALG, Libourne (FR)

(72) Inventors: Olivier CAGNAC, Libourne (FR); Axel ATHANE, Carbon Blanc (FR); Julien DEMOL, Les Billaux (FR)

(21) Appl. No.: 17/420,801

(22) PCT Filed: Jan. 10, 2020
(86) PCT No.: PCT/EP2020/050549

§ 371 Date: Jul. 6, 2021

(30) Foreign Application Priority Data
Jan. 11, 2019 (FR) 1900278

(51) Int. Cl. C07K 14/405; C07K 1/30; C07K 1/34; C12N 1/12

(52) U.S. Cl. CPC C07K 14/405; C07K 1/30; C12R 2001/89; C12N 1/12; C07K 1/34

(57) ABSTRACT

The invention relates to a new process for extracting and purifying phycocyanins produced by fermenting microalgae, in particular produced by Galdieria sulphuraria, by means of selective precipitation.

### FIELD OF THE INVENTION

The present invention relates to a novel process for extracting and purifying phycocyanins produced by fermentation of microalgae, in particular produced by Galdieria sulphuraria, by selective precipitation.

### PRIOR ART

The purification of phycobiliproteins extracted from Galdieria sulphuraria and Spirulina by ammonium sulfate precipitation has already been described in the literature (Moon et al., 2015; Cruz de Jesús et al., 2006, CN106190853), but it is very difficult to apply on an industrial scale because it requires a great deal of ammonium sulfate, which poses significant problems in reprocessing the ammonium sulfate and the supernatant.

The other purification methods described to obtain a high purity level, such as chromatography or ion-exchange resin purification (JP 2004359638), are very expensive to implement.

Precipitation of Spirulina phycocyanin has been described with the addition of acid to the crude phycocyanin solution (TN 2009000406, JP 2004359638, JP06271783, CN106190853). However, considering the isoelectric point of Spirulina phycocyanin, around 4.5 and close to the isoelectric point of a large number of other proteins, this method does not allow selective precipitation, and therefore purification of phycocyanin. Similarly, some describe the use of salicylic acid to precipitate phycocyanin from Spirulina (WO 2016/030643). The use of this acid generates a non-selective precipitation of PC and the resulting precipitate is particularly difficult to resolubilize. The same result was obtained with Galdieria PC.

Phycocyanin extraction processes generally consist in precipitating organic matter other than phycocyanins present in an aqueous crude extract from a microalgae fermentation to preserve phycocyanins in the supernatant, which will be filtered before precipitating phycocyanins (JP 2004359638). However, some organic compounds, particularly complex polysaccharides such as glycogen, remain soluble under the same conditions as phycocyanins.

In an industrial phycocyanin purification process, a filtration (ultrafiltration) step can be used to remove water in order to concentrate the phycocyanin and to remove small molecules (proteins, ions, organic acid, etc.) smaller than the cut-off threshold of the filter used, in order to obtain the purest phycocyanin possible. However, the cut-off threshold of the filter being lower than the size of the glycogen, it is not removed and increases the viscosity of the retentate, decreasing the filtration rates. The concentration-dependent viscosity effect of glycogen has been demonstrated using purified glycogen from Galdieria sulphuraria (Martinez-Garcia et al., 2017).

Moreover, the purified phycocyanins obtained retain high levels of these sugars, which may alter the qualities of the phycocyanins, in particular their coloring power, requiring the production and/or use of larger amounts of phycocyanins for the same effect. These residual polysaccharides act as a filler that adds to phycocyanin manufacturing costs and may limit the commercial uses of the resulting phycocyanin, for example in the preparation of foodstuffs with a low sugar content.

The aim is to improve processes for extracting and purifying phycocyanins extracted from biomass, both from a qualitative point of view and from an industrial and economic point of view, notably by reducing the residual sugar content in the final product, in particular the residual glycogen content.

### DISCLOSURE OF THE INVENTION

The process in accordance with the invention consists in performing a selective precipitation of the phycocyanins directly from the crude extract which contains them under conditions which respect the integrity of the phycobiliproteins and which allow the main impurities, in particular the polysaccharides including glycogen, to be maintained in solution.

This selective precipitation results from a combined action on two factors, simultaneously or sequentially in any order, on the one hand the pH of the solution and on the other hand the concentration of phycocyanin.

The process in accordance with the invention is particularly suitable for purifying acid pH-resistant phycocyanins produced by Galdieria sulphuraria.

### DETAILED DESCRIPTION OF THE INVENTION

The invention relates to a process for extracting phycocyanins from a solution comprising the phycocyanin(s), also called the initial phycocyanin solution, comprising a selective precipitation step which consists on the one hand in adjusting the pH of the initial solution to a selected value within a range of pH values in which the phycocyanins are less soluble (also called the instability range) and on the other hand in concentrating the phycocyanins in the solution to promote their precipitation, and then a step of recovery of the precipitated phycocyanin.

The two actions of pH adjustment and of concentration can be carried out simultaneously or sequentially, either by adjusting the pH of the initial solution before concentrating, or by concentrating the initial solution before adjusting the pH.

Surprisingly, the differential concentration conditions mean that only phycocyanins precipitate, the other products that can be described as impurities, particularly polysaccharides, remain in solution.

It is thus possible to recover the precipitated phycocyanins by separating them from the solution, and if need be to dry them to obtain a purified phycocyanin powder.

The process in accordance with the invention not only allows the extraction of phycocyanins from the solution, but also allows in the same step to purify them, the phycocyanins obtained being particularly pure with low residual sugar contents.

The process in accordance with the invention is particularly suitable for purifying a phycocyanin solution extracted from a phycocyanin-producing microorganism culture which also produces glycogen, in particular in the context of an industrial phycocyanin production process which comprises culturing the microorganisms, then recovering the biomass produced to extract the phycocyanin, and recovering the phycocyanin from this biomass.

The process is particularly suitable for phycocyanins produced by microorganisms that produce high levels of glycogen, especially for extracting and purifying phycocyanins from biomass that comprises more than 10% glycogen based on total dry matter.

Phycocyanin-producing microorganisms are well known, including algae (or microalgae) of the order Cyanidiales. The order Cyanidiales includes the families Cyanidiaceae or Galdieriaceae, themselves subdivided into the genera Cyanidioschyzon, Cyanidium or Galdieria, to which belong among other species Cyanidioschyzon merolae 10D, Cyanidioschyzon merolae DBV201, Cyanidium caldarium, Cyanidium daedalum, Cyanidium maximum, Cyanidium partitum, Cyanidium rumpens, Galdieria daedala, Galdieria maxima, Galdieria partita or Galdieria sulphuraria. Particular mention may be made of the strain Galdieria sulphuraria (also known as Cyanidium caldarium) UTEX 2919.

Mention may also be made of known phycocyanin producers such as the filamentous cyanobacteria of the genus Arthrospira, which are industrially cultivated under the common name of spirulina.

The microorganisms that produce phycocyanin with a high glycogen content are particularly identified among the microorganisms mentioned above, especially species of the genera Cyanidioschyzon, Cyanidium and Galdieria, more particularly Galdieria sulphuraria.

Industrial processes for culturing phycocyanin-producing microorganisms are well known to the person skilled in the art. Particular mention may be made of patent applications WO 2017/093345, WO 2017/050917.

The recovery of phycocyanin from biomass is also known to the skilled person. Particular mention may be made of patent application WO 2018/178334. It generally requires a step of cell, mechanical or enzymatic lysis in order to release the produced phycocyanin in the cellular compartments of the microorganisms. This lysis is advantageously carried out at a pH favorable to the solubilization of phycocyanins. This cell lysis will generally generate a phycocyanin solution which comprises organic matter in suspension (called crude suspension) which can be separated by usual filtration methods. A crude phycocyanin solution is then obtained which can be further purified to remove low molecular-weight organic residues by usual ultrafiltration methods to obtain a refined solution from which phycocyanin can be obtained by usual precipitation and drying methods. Particular mention may be made of tangential filtration on ceramic membranes or organic membranes such as polyethersulfone hollow fibers. The thresholds of these filters can be chosen to separate molecules of molecular weight higher or lower than the targeted phycobiliproteins.

The process in accordance with the invention is particularly suitable for purifying acid pH-resistant phycocyanin solutions, in particular the phycocyanins described in application WO 2017/050918.

In particular, the process in accordance with the invention is used for purifying acid pH-resistant phycocyanins produced by Galdieria sulphuraria, more particularly in an industrial process for producing these phycocyanins by fermenter culture of Galdieria sulphuraria.

The process is advantageously implemented to extract phycocyanin from the crude juice obtained from a phycocyanin-producing microorganism biomass.

Advantageously, the initial phycocyanin solution, in particular the crude juice, comprises from 0.1 to 10 g/L of phycocyanin.

Concentration consists in removing water so as to obtain a phycocyanin content of at least 15 g/L, preferably at least 20 g/L, more preferentially at least 30 g/L, or even at least 40 g/L.

This concentration can be defined as % volume loss based on the phycocyanin content in the initial solution.

In an industrial phycocyanin production process, the crude juice will advantageously comprise at least 1 g/L of phycocyanins. In this case, concentration will consist in removing at least 93% of the initial volume of liquid.

Concentration is done by any method known to allow the removal of water under conditions that preserve the integrity of phycocyanins. Mention may be made of water evaporation methods, in particular under reduced pressure to promote this evaporation under temperature conditions that respect the integrity of phycocyanins, without affecting their coloring power. Mention may also be made of methods that allow the removal of a liquid, such as tangential filtration with pore sizes that allow water and small molecules in solution to pass but retain proteins.

These filtration methods and the devices for carrying them out are well known to the person skilled in the art, in particular the Spectrum Labs TFF systems from Repligen. For phycocyanins, it is advantageous to choose filters with pores of 50 kD to 100 kD, in particular polyethersulfone or polysulfone filters.

The pH adjustment consists in adding an acid or a base to the initial solution or to the concentrated solution in order to reach a pH value in the instability range. The instability range will depend on the phycocyanins to be purified, and in particular on the microorganism that produced it. In general, this instability range is from 4.5 to 5.5, in particular for acid pH-resistant phycocyanins as described above.

For these acid pH-resistant phycocyanins, cell lysis is done at acidic pH, preferably below 4.5, usually about 4, or even down to 3.

The pH adjustment then consists of adding a base to reach the pH in the instability range.

According to a first embodiment of the invention, the process first consists in concentrating the initial juice. In this case, the concentration is carried out at a pH favorable to the solubilization of the phycocyanins, i.e., outside the instability range. For the acid pH resistant phycocyanins described above, these pH favorable to the solubilization of phycocyanins will advantageously be lower than 4 or higher than 5.

According to another preferred embodiment of the invention, the process consists first in adjusting the pH to the instability range and then concentrating the solution until the phycocyanins precipitate.

The process can then be described as obtaining a solution of unstable pH from an initial solution, then concentrating the solution of unstable pH to cause the precipitation of phycocyanins. The percentage of volume reduction will be reached when the precipitation of phycocyanins is observed.

This selective precipitation step is advantageously carried out at room temperature. Of course, the person skilled in the art will be able to modify the temperature in such a way as to favor precipitation, for example by lowering the temperature to implement the second part of the step (concentration or pH adjustment) during which precipitation takes place.

Polysaccharides in solution, in particular glycogen, can then be recovered by usual polysaccharide precipitation methods, for example by addition of ethanol (Martinez-Garcia et al., 2016), which polysaccharides can also be subsequently purified.

According to a particular embodiment of the invention, the polysaccharides contained in the initial solution with the phycocyanins are subjected to enzymatic lysis which favors their retention in solution. The trace amounts of these polysaccharides likely to be carried away with phycocyanin precipitation, which are already low, are reduced even further when the polysaccharides are lysed into low-molecular-weight oligosaccharides which are even more soluble. Moreover, when the concentration step is carried out by tangential filtration, the low-molecular-weight oligosaccharides are removed with the other small molecules in solution, which favors the obtaining of a solution with an even higher phycocyanin content.

In particular, enzymatic lysis of glycogen is carried out at a pH of less than or equal to 5, preferably of about 4.5, at room temperature.

These temperature and pH conditions are particularly suitable to preserve the phycocyanin during the enzymatic reaction.

Enzymes active under acidic pH conditions and at room temperature are selected from enzymes known to have α1-4 glucuronidase, α1-4 glucosidase (or alpha glucosidase) activity. Particular mention will be made of pectinases known to degrade pectin and in particular pectinases extracted from filamentous fungi such as Aspergillus, more particularly pectinases extracted from Aspergillus aculeatus, such as the enzymes marketed under the name Pectinex® by the company Novozymes.

Enzymatic lysis of glycogen could also be achieved with an α1-6 glucosidase in addition to α1-4 glucuronidase or α1-4 glucosidase. α1-6 Glucosidases active under the pH and temperature conditions set forth above are also known to the skilled person. In particular, these are pullulanases known to hydrolyze α1-6 glycosidic bonds of pullulan, in particular known to remove starch branches.

These are generally enzymes extracted from bacteria, particularly from the genus Bacillus. U.S. Pat. Nos. 6,074,854, 5,817,498 and WO 2009/075682 describe such pullulanases extracted from Bacillus deramificans or Bacillus acidopullulyticus. Commercially available pullulanases are also known, notably under the names “Promozyme D2” (Novozymes), “Novozym 26062” (Novozymes) and “Optimax L 1000” (DuPont-Genencor).

It will be noted that pullulanase/alpha-amylase mixtures are described in the prior art, but in particular to produce glucose syrup from starch (US 2017/159090).

The skilled person will know how to determine the appropriate reaction conditions to best reduce the amount of glycogen as a function of the initial glycogen content in the solution to be treated, the amount of enzymes used and the desired purity of the phycocyanin produced.

The recovery of solid precipitated phycocyanins is done by any method known to the skilled person, such as filtration or centrifugation.

The skilled person will be able to envisage any method of recovery of the solids so as to reduce the volume to be treated by filtration or centrifugation.

This recovery can be done discontinuously, in batches, or continuously, with the addition of initial solution to compensate for the recovery of solid phycocyanins.

This continuous recovery step will advantageously be implemented with a concentration by tangential filtration on a solution of unstable pH, the person skilled in the art being able to adjust the respective flow rates of water removal and supply of solution of unstable pH to promote the precipitation of phycocyanins.

Such a continuous process will be particularly adapted to treat initial solutions in which polysaccharides and in particular glycogen will have undergone an enzymatic lysis which favors their removal by tangential filtration with water and the other small soluble molecules.

The invention also relates to a process for producing phycocyanins by fermentation of microorganisms, said process comprising the following steps of (i) cultivating the microorganisms to obtain a phycocyanin-rich biomass, (ii) recovering the biomass and cell lysis to solubilize the released phycocyanins in a suspension of cell particles, (iii) clarifying the previously obtained suspension to obtain a crude phycocyanin solution and (iv) recovering the phycocyanin from the previously obtained crude solution, characterized in that the recovery of the phycocyanin comprises a selective precipitation step as defined above.

The recovered solid can then be dried by any suitable method and, if need be, ground.

The recovered solid comprising phycocyanin can also be subjected to purification by methods known to the skilled person, such as diafiltration.

The methods of fermentation cultivation, biomass recovery, lysis and clarification are well known to the skilled person, in particular those described in patent applications WO 2017/050917, WO 2017/093345 and WO 2018/178334.

The selective precipitation implemented in accordance with the invention globally decreases the energy necessary to produce phycocyanin powder from an initial solution, in particular from a crude juice, both in terms of the amount of material to be handled and the energy necessary to dry the solid phycocyanin and grind it.

The phycocyanin obtained by this process has a purity index of at least 2, preferably at least 3, or even higher than 4.

This purity index is measured by absorbance measurement with the method described by Moon et al. (2014).

Advantageously, the phycocyanin obtained is a phycocyanin which has a glycogen/phycocyanin ratio (by dry weight) lower than 6, advantageously lower than 4, preferably lower than 3, more preferentially lower than 2.5, even more preferentially lower than 1.

The invention also relates to the use of the phycocyanins obtained as colorants, in particular as food colorants. It also relates to solid or liquid foods, in particular beverages, which comprise a low-glycogen phycocyanin in accordance with the invention.

### DESCRIPTION OF THE FIGURES

FIG. 1 shows the mass of the precipitate obtained at different phycocyanin concentrations and different pHs in the initial solution.

FIG. 2 shows the phycocyanin concentration in the supernatant after precipitate recovery as a function of pH for different phycocyanin concentrations.

### EXAMPLES

Material and Methods

Strain: Galdieria sulphuraria (also called Cyanidium caldarium) UTEX #2919.

Cultivation Conditions:

The biomass is obtained by fed-batch fermentation using the conditions described in patent WO2017050918A1.

Extraction Conditions:

The cells are mechanically ground using a DYNO®-MILL KD ball mill (Willy A. Bachofen AG Maschinenfabrik). Since phycocyanin (PC) is a hydrophilic molecule, it is extracted with water by adjusting the pH to the desired value with a base (NaOH, KOH, NH₄OH, etc.) or an acid (H₂SO₄, citric acid, etc.). The crude PC extract is recovered after separation of cell debris by centrifugation at 10000 g for 10 min at room temperature. The crude extract is concentrated by tangential filtration with a ceramic or organic membrane with a cut-off threshold allowing phycocyanin to be retained. The samples are then centrifuged to separate the precipitate from the supernatant. The mass of the pellet is measured with a precision balance. The pellet is resuspended in an aqueous solution of pH 7 allowing its resolubilization, in order to quantify the precipitated phycocyanin.

PC Determination:

The estimation of phycocyanin content and of purity index was performed by absorbance measurement using the method described by Moon et al. (Moon et al., Korean J. Chem. Eng., 2014, 1-6).

### EXAMPLE 1

### Effect of Concentration and pH on the Precipitation and Purification of Phycocyanin (PC)

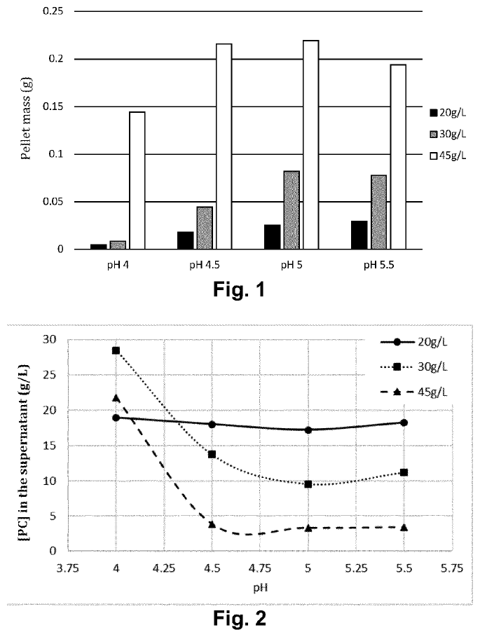

A crude phycocyanin solution with an initial concentration of 1 g/L of PC and an initial purity of 1.6 is concentrated by tangential filtration at pH 4 to obtain a retentate with a concentration of 20 g/L, then 30 g/L and then 45 g/L. An increase in the purity of the product can be observed during the filtration, however this purity does not exceed the value of 2 despite the degree of concentration of the product. In FIG. 1 it can be seen that for a concentration of 20 g/L, the precipitation of phycocyanin is low at pH 4 and increases slightly as the pH increases towards higher values (FIG. 1). At the same time, the measurement of the soluble phycocyanin concentration in the supernatant during this pH rise shows a relatively small decrease. For a PC concentration of 30 g/L, this phenomenon of precipitation by change of pH is much more marked and appears to be maximal for values of 4.5 and 5.5 (FIG. 1 and FIG. 2). Upon further filtration and concentration of phycocyanin up to the value of 40 g/L soluble, the formation of a significant precipitate is observed during filtration even before pH modification (FIG. 1). As before, the change in pH increases the phenomenon of phycocyanin precipitation.

By tangential filtration, the purity of phycocyanin decreases and conversely the purity of the precipitate collected and resolubilized at pH 7. This indicates a preferential precipitation of phycocyanin, which can be resolubilized under more favorable pH conditions.

Table 1 reports the measurement of phycocyanin purity after resolubilization of the phycocyanin precipitate for precipitation at pH 7.5.

TABLE 1

| PC concentration | PC purity of | PC purity of pellet |
|---|---|---|
| in the sample | the supernatant | resuspended at pH 7.5 |
| 20 g/L | 1.96 | 2.18 |
| 30 g/L | 1 | 2.27 |
| 45 g/L | 0.31 | 2.88 |

### EXAMPLE 2

### Effect of Concentration and pH on the Precipitation and Purification of PC from an Enzymatically Digested Sample

In this example, the crude solution is subjected to enzymatic digestion to degrade the glycogen present. The enzymatic degradation is done at room temperature and pH=4 with the enzymes alpha 1-4 glucuronidase (“Pectinex Ultra SPL”) and alpha 1-6 glucosidase (“Novozyme 26062”).

Enrichment by tangential filtration is carried out to reach a phycocyanin concentration of several tens of g/L and then a pH adjustment is performed, causing precipitation. pH samples at 4.5, 5 and 5.5 are taken with a measurement of residual soluble phycocyanin and a measurement of precipitated phycocyanin after collection and resolubilization of the pellet with a buffer solution at pH 7.5.

Similar to the previous example, precipitation is significant for a pH range comprised between 4.5 and 5.5, and the purity level in this case reaches values above 3.8 upon resolubilization of the precipitated phycocyanin. Enzymatic digestion of glycogen does not affect the purification by precipitation. Table 2 below gives the purity index values of phycocyanin after precipitation by adjustment to instability pH and also after resolubilization of the phycocyanin precipitate.

TABLE 2

| Sample | [PC] (g · L⁻¹) | Purity |
|---|---|---|
| Supernatant pH 4.5 | 5.90 | 1.08 |
| Supernatant pH 5 | 7.19 | 1.25 |
| Supernatant pH 5.5 | 10.79 | 1.51 |
| Pellet pH 4.5 | 22.26 | 3.82 |
| Pellet pH 5 | 19.82 | 3.90 |
| Pellet pH 5.5 | 16.55 | 3.87 |

### REFERENCES

Cruz de Jesùs et al., Int J Food Nutr Sci (2016) 3(3): 1-0
Martinez-Garcia et al., Int J Biol Macromol. (2016) 89:12-8
Martinez-Garcia et al., Carbohydrate Polymers (2017) 169: 75-82
Moon et al., 2014 Korean J. Chem. Eng., 2014, 1-6
TN 2009000406, U.S. Pat. Nos. 6,074,854, 5,817,498, US 2017/159090, WO 2009/075682, WO 2016/030643, WO 2017/050917, WO 2017/050918, WO 2017/093345, WO 2018/178334

1. A process for extracting phycocyanins from an initial phycocyanin solution, wherein the process comprises the following steps:
a) selective precipitation which consists in:
(i) adjusting the pH of the initial solution to a selected value within a range of pH values in which the phycocyanins are less soluble,
(ii) concentrating the phycocyanins in the solution to promote their precipitation, and
b) recovery of the precipitated phycocyanin.

2. The process according to claim 1, wherein the steps (i) of pH adjustment and (ii) of concentration are carried out simultaneously or sequentially.

3. The process according to claim 1, wherein the initial phycocyanin solution is a crude solution obtained from a lysis of a microorganisms biomass cultivated to produce phycocyanin.

4. The process according to claim 1, wherein the phycocyanin is a phycocyanin stable at acidic pH.

5. The process according to claim 1, wherein the phycocyanin is a phycocyanin of microbial origin, produced by a microorganism selected from species of the genus Cyanidioschyzon, Cyanidium or Galdieria.

6. The process according to claim 1, wherein the range of pH values in which the phycocyanins are less soluble is from 4.5 to 5.5.

7. The process according to claim 1, wherein the concentration consists in removing water so as to obtain a phycocyanin content of at least 15 g/L.

8. The process according to claim 1, wherein the concentration step is carried out by tangential filtration with a cut-off threshold allowing the phycocyanin to be retained.

9. The process according to claim 1, wherein the recovered phycocyanin is dried and optionally ground.

10. The process according to claim 1, wherein the recovered phycocyanin has a purity index of at least 2.

11. The process according to claim 10, wherein the recovered phycocyanin has a purity index higher than 4.

12. A purified phycocyanin obtained by the process according to claim 1.

13.-14. (canceled)

15. The process according to claim 2, wherein the step (i) of pH adjustment is carried out before the step (ii) of concentration.

16. The process according to claim 3, wherein the microorganism also produces glycogen.

17. The process according to claim 16, wherein the obtained phycocyanin is a phycocyanin having a glycogen/phycocyanin weight ratio lower than 6.

18. The process according to claim 1, wherein the recovered phycocyanin has a purity index of at least 3.
